(19) United States
(12) Patent Application Publication
(10) Pub. No.: US 2006/0122472 A1
(43) Pub. Date: Jun. 8, 2006

(54) System and method for clinically assessing motor function

(76) Inventor: Seth L. Pullman, Scarsdale, NY (US)

Correspondence Address: BAKER & BOTTS, 30 ROCKEFELLER PLAZA, NEW YORK, NY, 10112, US

(21) Appl. No.: 11/237,539

(22) Filed: Sep. 28, 2005

Related U.S. Application Data
(60) Provisional application No. 60/614,033, filed on Sep. 28, 2004.

(51) Int. Cl. A61B 5/00

(52) U.S. Cl. 600/300

(57) ABSTRACT

The present invention relates to methods and systems for assessing and diagnosing motor conditions using indices derived from computational analysis of a digitized drawing sample of a spiral. It is an improvement of the invention disclosed in U.S. Pat. No. 6,454,706, and adds new indices and depictions thereof. Such indices may be used individually or in combinations amongst themselves or with indices disclosed in U.S. Pat. No. 6,454,706 to evaluate and assess motor function. As such, in non-limiting embodiments, the new information may be used in diagnostic techniques relative to an expert generated standard or, alternatively, may be used to monitor a subject's function over time or as a result of treatment, where indices or their combinations are compared between drawing samples obtained from a single subject or a group of subjects. The methods and systems of the invention have application in both the clinical and research communities.

This application claims priority to U.S. Provisional Application No. 60/614,033, filed Sep. 28, 2004, which is incorporated by reference in its entirety herein.

A portion of the disclosure of this patent document contains material which is subject to copyright protection. The copyright owner has no objection to the facsimile reproduction by anyone of any portion of the patent document, as it appears in any patent granted from the present application or in the Patent and Trademark Office file or records available to the public, but otherwise reserves all copyright rights whatsoever.

### INTRODUCTION

This invention relates to the objective assessment of motor function by computer analysis of a digitized drawing sample of a spiral, as may be used in the diagnosis and monitoring of motor disorders (including tremor) as well as the evaluation of motor development and handedness in children.

### BACKGROUND OF THE INVENTION

A patient may seek medical treatment for a variety of complaints which suggest a disturbance of motor function, such as weakness, stiffness, tremor, clumsiness, or difficulty in executing movements. It then is the physician's responsibility to correctly diagnose the patient, and to implement the appropriate course of treatment. A number of syndromes which involve motor dysfunction exist, and are defined by their clinical manifestations.

For example, Parkinson's Disease, which results from a degeneration of cells in the basal ganglia of the brain, is associated with slowness of movement (“bradykinesia”), muscle rigidity, and a tremor often said to have a “pill rolling” quality which occurs at rest but tends to diminish with voluntary movements. In addition, patients suffering from Parkinson's Disease may exhibit a loss of facial expression, a difficulty in initiating movements, and a diminution of their handwriting (“micrographia”).

Another fairly common motor disorder is essential tremor, an inherited condition which can present in childhood but more typically appears later in adult life. It usually involves the upper limbs, but may also affect the head, jaw, lips, tongue and pharynx. This tremor may abate upon ingestion of alcohol or beta-adrenergic antagonists. It may interfere with voluntary movements to the point where a sufferer is unable to drink from a glass or raise a spoon without spilling its contents.

There are numerous other motor disorders from hyperkinetic conditions such as essential tremor mentioned above to complex akinetic-rigid and other degenerative syndromes. Motor disorders may be considered primary when there are no known causes (other than genetics) and secondary, or symptomatic, when a known etiologic agent exists. Examples of primary motor disorders include Parkinson's disease, essential tremor and adult onset focal dystonia such as writer's cramp. Secondary motor disorders are more numerous and include Parkinsonian syndromes, side effects of medications such as tardive dyskinesia from neuroleptic use, immune, ischemic or even traumatic causes.

The multitude of motor disorders share many overlapping symptoms and signs. Even though sophisticated rating systems have been developed for some disorders (e.g., Parkinson's Disease) to aid in the accuracy of diagnosis, in the hands of inexperienced practitioners, or where the disease is in its early stages and clinical signs are subtle, the potential for an erroneous diagnosis is substantial. Diagnosis by a traditional neurologic exam may also be difficult where the patient is unable to comply with fairly detailed instructions for tests used to evaluate motor function. As an example, a young child or a demented adult suspected of having a defect in motor development may be difficult to evaluate.

If an error in diagnosis is made, there may be significant adverse consequences. For example, the appropriate therapies for Parkinson's Disease and essential tremor are very different, in that patients with Parkinson's Disease are treated with agents that increase or facilitate dopamine activity whereas patients with essential tremor are treated with agents that block beta-adrenergic neurotransmitters. Not only would misdiagnosis result in a lack of a clinical benefit, but administering the inappropriate drug could have undesirable or even toxic side effects.

For example, beta adrenergenic blocking agents can adversely affect cardiac or pulmonary functions; unnecessary use in a Parkinson's Disease patient, particularly an older patient, could be dangerous. Similarly, use of agents that treat Parkinson's disease in a patient without that condition could have harmful consequences. Specific examples of Parkinson's Disease treating agents include artane, sinemet and baclofen. Artane, an anticholinergic agent used to treat Parkinsonian tremors and dystonia can severely affect cognition, cardiac, visual and urinary function. Sinemet, a mainstay drug for Parkinson's disease, causes nausea, vomiting, hallucinations and low blood pressure. Baclofen, an anti-spasmodic agent, and clonazepam, an anxiolytic and muscle relaxant, are used in many motor disorders but can alter mental status, blood pressure and can even be fatal when used inappropriately.

Further, even where the correct diagnosis has been made, it is important to be able to evaluate the clinical progress of a patient. Often the methods for measuring progress are extremely subjective.

One means by which clinicians have attempted to decrease subjectivity in diagnosis and monitoring motor function has been through the use of standardized clinical tests. Examples of such tests include asking the patient to touch his finger to, alternately, his nose and the outstretched finger of the examiner, or to run her heel up and down her shin, or to touch his or her thumb to, in succession, each of the other fingertips of the same hand.

Drawing has been used to evaluate motor function for many years. The famous neuropsychiatrist Kraepelin, at the beginning of this century, adapted an instrument to quantitatively analyze signatures for the evaluation of motor function in schizophrenic patients (Blyler et al., 1997, Schizophrenia Res. 26: 15-23, citing Hoch, 1904, Psychol. Bull. 1:241-257). One common test involves asking the patient to draw an Archimedes spiral. A thorough discussion of the spiral drawing test may be found in Bain & Findley, in “Standards in Neurology, Series A: Assessment, diagnosis and evaluation, Book I: Assessing Tremor Severity,” published by Smith Gordon and Co., Ltd., London, England/Nishimura Co., Ltd., Niigata-Shi, Japan, copies of which can be obtained in the United States through Books International Inc., Herndon, Va. According to that reference, the severity of tremor apparent in the spiral is rated from 0-10, where critical factors in determining the grade of a particular spiral are the degree of perpendicular displacement of the track from the intended trajectory and the extent to which tremor persists during each turn (Bain & Findley, p. 9). Tremor is said to become more apparent in the outward turns of the spiral. An example of a study which used spiral analysis to quantify the effects of the drug terguride in Parkinson's Disease patients is reported in Filipová et al., 1988, Eur. Arch. Psychiatr. Neurol. Sci. 237:298-303. Another study which used spiral copying ability to evaluate the effect of the drug ondasetron on cerebellar tremor is described in Rice et al., 1997, J. Neurol. Neurosur. & Psychiat. 62:282-284.

A number of investigators have attempted to lessen the subjectivity of evaluation by using computer assistance. For example, Elble et al. (1996, Movement Disorders 11:70-78) asked patients with essential tremor to write a series of cursive e's and 1's and, in some cases, to draw an Archimedes spiral on a digitizing tablet. They reported detecting changes in mean acceleration amplitude and tremor frequency with an accuracy which indicated that use of such a tablet was an accurate and less-costly alternative to accelerometry for tremor evaluation.

Wissel et al. (1996, J. Neurol. Neurosurg. & Psychiat. 61:172-175) used a digitizing tablet to measure writing speed in an evaluation of the effectiveness of botulinum toxin for treating writer's cramp.

Eichorn et al., 1996, (Movement Disorders 11:289-297) used a computational analysis of open loop handwriting movements, as captured by a digitizing tablet, to monitor the effect of apomorphine on patients with early untreated Parkinson's Disease. They reported that computer-assisted analysis of automated handwriting movements can be a quick method for quantifying dopamimetic effects on handwriting movements in parkinsonian patients. However, they also found that there was no statistically significant correlation when changes in the individual handwriting parameters were correlated with a subscore obtained using the Unified Parkinson's Disease Rating Scale (“UPDRS”; Lang and Fahn, 1989, in “Quantification of neurologic deficit,” Munsat, ed., Butterworth-Heinemann, Storeham, Mass., pp. 285-309) for the writing hand, an observation which they indicated was expected, as the UPDRS assesses different kinds of parkinsonian symptoms, such as rigidity, akinesia, and tremor.

Blyler et al. (1997, Schizophrenia Res. 26: 15-23) used line drawing to measure lateralized motor performance in schizophrenic patients. The patients drew lines on a piece of paper, which were then scanned into a computer and a regression was run on the points of the line and used to calculate the deviation from straightness. The results were found to correlate with clinical rating scales of motor function, including the Simpson-Argus Rating Scale (Simpson and Argus, 1970, Acta Psychiatr. Scand. 212 (Suppl.), 9-11) for parkinsonian symptoms.

Slavin et al., 1999, J. Internatl. Neuropsychol. Soc. 5:20-25, used a digitizing tablet to analyze writing samples from patients with dementia of Alzheimer's type (“DAT”). Kinematic measures of stroke length, duration, and peak velocity were expressed in terms of consistency via a signal-to-noise ratio. Patterns typical of DAT but not Parkinson's disease were observed.

Lange-Küttner (1998, Perceptual and Motor Skills 86:1299-1310) report that speeded drawing of basic graphic patterns by young children, as captured on a digitizing tablet, could be used to identify psychophysical problems.

Computational analysis of handwriting, for identification or analytical purposes, is described in Singer and Tishby, 1994, Biol. Cybern. 71: 227-237; van den Heuvel et al., 1998, Acta Psychologica 100: 145-159; and Morasso and Sanguineti, 1993, Acta Psychologica 82: 213-235.

U.S. Pat. No. 6,454,706 by Pullman, issued Sep. 24, 2002, incorporated by reference in its entirety herein, relates to methods and systems for assessing motor function in which a subject draws a geometric pattern such as a spiral. The drawing is converted into digital information, which is then interpreted to derive various indices which reflect characteristics of motor performance. Indices may be used, in conjunction with an expert generated standard of reference, to compute a clinical rating score that may be used toward assessing and/or diagnosing the subject's motor condition. The present invention is an improvement of the invention set forth in U.S. Pat. No. 6,454,706.

### SUMMARY OF THE INVENTION

The present invention relates to methods and systems for assessing and diagnosing motor conditions using indices derived from computational analysis of a digitized drawing sample of a spiral. It is an improvement of the invention disclosed in U.S. Pat. No. 6,454,706, and adds new indices and depictions thereof. Such indices may be used individually or in combinations amongst themselves or with indices disclosed in U.S. Pat. No. 6,454,706 to evaluate and assess motor function. As such, in non-limiting embodiments, the new information may be used in diagnostic techniques relative to an expert generated standard or, alternatively, may be used to monitor a subject's function over time or as a result of treatment, where indices or their combinations are compared between drawing samples obtained from a single subject or a group of subjects. The methods and systems of the invention have application in both the clinical and research communities.

### DESCRIPTION OF THE FIGURES

FIG. 1 is a hardware block diagram of a system for analyzing movement disorders in accordance with a preferred embodiment of the present invention;

FIG. 2 is a software block diagram corresponding to the system of FIG. 1;

FIG. 3 is an illustration of an electronic tablet in accordance with a preferred embodiment of the present invention;

FIG. 4A-L presents depictions of clinical data obtained from a subject.

FIG. 5A-B shows correct (A) and incorrect (B) arm position for drawing spirals.

FIG. 6A-B illustrate a non-limiting example of the main window (A) and drawing window (B) of a digital tablet, showing a drawn spiral.

FIG. 7 illustrates a non-limiting example of the main window and display associated with saving recorded data.

FIG. 8 illustrates a non-limiting example of the contends of a “raw data” sub-folder containing data associated with ten spirals drawn by a subject's right hand and ten spirals drawn by a subject's left hand.

FIG. 9A-G illustrates non-limiting examples of (A) a menu for selecting and opening a “Spiral Analysis” program; (B) a window with programming code; (C) a “Spiral Analysis Interface” screen which allows input as to whether the subject is a patient or a control, and whether all spirals, an individual spiral, or a partial set of spirals are analyzed; (D) a menu for analyzing all or a subset of spirals; (E) a menu for deselecting particular spirals; (F) a display showing spirals cut to eliminate beginning and ending portions; and (G) a display showing cross-hairs for selecting portions of a spiral to be cut.

FIG. 10A-C illustrates non-liming examples of (A) a menu showing parameters/values; (B) a menu for selecting trials to be analyzed; and (C) a window relating to printing mode.

FIG. 11A-C illustrates non-limiting examples of (A) an individual trial page with degree of severity and other spiral indices (in a preferred embodiment, there may be 20 such pages): (B) a summary figure showing all tremor axes for all spirals collected, number of axes per trial, ratio of last over first spiral speed segments and graph superimposing all spiral speed curves; and (C) a summary sheet showing the most commonly used results, with comparisons to normative data.

### DETAILED DESCRIPTION OF THE INVENTION

For clarity of description, and not by way of limitation, the detailed description of the invention is divided into the following subsections:

i) acquisition of spiral data;

ii) analysis of spiral data; and

iii) uses of the invention.

### Acquisition of Spiral Data

The present invention utilizes a surface that is capable of converting the spiral drawing made by the subject into digital information that may be subjected to analysis. In preferred embodiments, the surface is comprised in a writing tablet, but other embodiments would include, but not be limited to, a surface on a desk, table, or wall (e.g., a digitized blackboard). Optionally, a piece of paper may be applied to the surface to subjectively aid the subject in drawing, or the surface itself may have paper-like characteristics. A suitable instrument may be any implement that can function in conjunction with the surface to produce digital data, and may be configured to resemble a conventional pen or pencil, and is preferably cordless.

In alternative embodiments of the invention, the subject may or may not be able to observe the drawing of the spiral. Drawing the spiral “blind” would eliminate the subject's ability to compensate, based on visual feedback, for irregularities in the drawing. Further, in embodiments where the subject is able to observe the drawing process, observation may be direct or indirect. Direct observation would include being able to passively observe the drawing process. Indirect observation would include watching the drawing process via a monitor or a mirror, so that the observed image would be, in certain embodiments, a mirror image of the act of drawing.

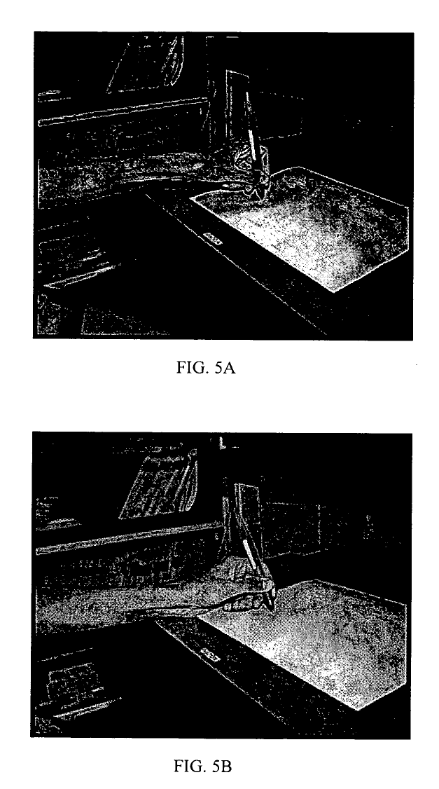

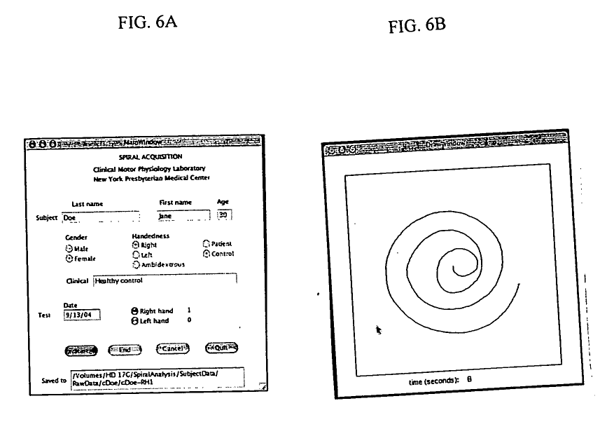

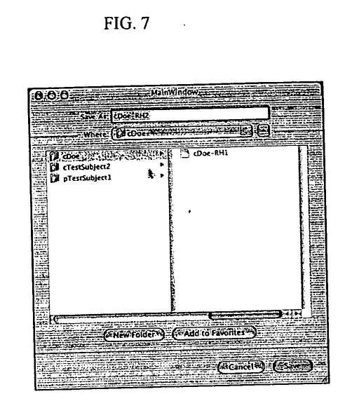

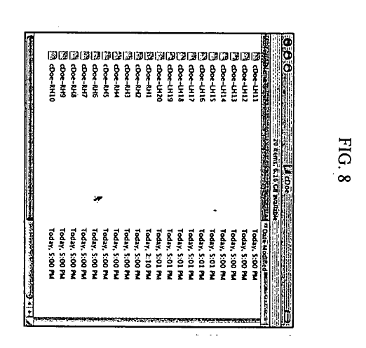

A preferred, specific, non-limiting embodiment of the drawing process is as follows. Medical history information of a test subject is recorded, such as first and last name, age, gender, handedness, whether the subject is subject to diagnosis or is a “healthy” control, and clinical information such as possible diagnosis, treatment, and relevant historical facts. The subject is shown how to draw a spiral on a digitized tablet as described herein, starting at a set point (“X”) and drawing at moderate speed with the wrist held off the tablet, and the elbow controlled by the upper arm. Keeping the wrist off the tablet allows the subject's hand to move freely, so that they are not just rotating their hand with their wrist anchored to the tablet. The elbow should be off the tablet as well, with the forearm approximately level with the tablet. Correct and incorrect hand positions are shown in FIGS. 5A and 5B. The subject is then asked to draw ten spirals with his or her right hand, and then 10 spirals with his or her left hand, on the a digitized tablet as described herein. The subject is told when to start and when to stop drawing, and upon the “stop” command, they should lift the pen off the tablet. As or after the subject has started drawing a spiral, the device is activated into a data acquisition mode. FIGS. 6A and 6B show non-limiting examples of a main window and draw window. People draw at varying speeds; if the subject draws very quickly, it may be desirable to ask them to lift the pen as they reach the edge of the box, because people will often slow down “artificially” thinking that they need to stay within the lines. Alternatively, if the subject draws very slowly, it may be desirable to record not more than 20 seconds of information, even if they have not reached the edge of the box, as collecting more information may overburden the analysis system. That said, a minimum of three 360-degree rotations (loops of the spiral) are preferably for analysis. If this condition is satisfied in fewer than 4 seconds, it may be desirable to record to 4 seconds. If a problem is encountered in data acquisition, it may be desirable to cancel pre-existing data and then restart the tablet. If a tablet such as a Wacom tablet is used, there may be a cap on the pressure that can be detected by the tablet. If the cap is exceeded (for example, if the patient is pressing too hard), the program may indicate this by recording data collected in red. Sometimes, depending on the patient's disorder, using too much pressure cannot be avoided. To stop recording data, it is preferable to initiate deactivation of the data acquisition mode a “split second” before asking the subject to stop drawing. The data may then be saved, for example to an assigned file name, e.g. a name that incorporates the last name of the subject. FIG. 7 provides a non-limiting illustration of the main window of a device during the save step. If, for some reason, the default name becomes incorrect, for example, if “left hand” is not timely selected when switching the same subject to left handed spirals, then either (i) changes can be made during the process (in the specific software provided herein, each filename must be in the exact format cDOe-RH2, where c designates “control” (alternatively, a “p” for patient is used) and Doe indicates the position of the last name of the subject, RH=right hand, LH=left hand, the numbers 1-10 are used for RH, and the numbers 11-20 are used for LH; or (ii) changes can be made after finishing data collection but before analysis (sorting may be performed using “time modified” to see which spirals were done first/later if the number or RH/LH designation needs to be corrected after the fact). After ten spirals are drawn (saved as RH1-RH10) with the right hand, the patient may be asked to switch to his or her left hand, and the device may be signaled (e.g., by pushing a “left hand” button on the main window box) that the left hand is being used to draw subsequent spirals. Recording then may be performed as set forth above. After the desired number (e.g., 20) of spirals has been drawn, the data acquisition program may be terminated, for example by hitting a “quit” button. For example, and not by way of limitation, the data may be saved in a “raw data” folder having sub-folders that contain data from particular subjects; once it is confirmed that all the relevant spirals produced in the session are accounted for (see FIG. 8 for a non-limiting illustration) the sub-folder containing the raw data may be duplicated to be saved on a Spiral Analysis Back-up Zip Disk.

An example of Spiral Acquisition Data is comprised herein as Appendix A. A Spiral Acquisition Program is included in Appendix B, supplied on diskette.

### Analysis of Spiral Data

The present invention provides for a system for clinically assessing motor function in a subject that includes: an electronic digitizing tablet having a writing device for obtaining a spiral pattern handwritten by the subject and providing one or more digital signals representing the pattern; and a microprocessor for processing the signals to derive one or more geometric indices representative of motor function and for computing from the indices, using an expert-generated “standard of reference”, a clinical rating score indicative of motor function of the subject.

In another aspect of the present invention, a preferred method for analyzing movement disorders includes: a method for clinically assessing motor function in a subject comprising: obtaining a spiral pattern handwritten by the subject on a digitizing tablet; generating one or more digital signals representing the geometric pattern; processing the signals to derive one or more geometric indices representative of motor function; and computing from the geometric indices, using the aforementioned expert-generated “standard of reference”, a clinical rating score indicative of motor function of the subject.

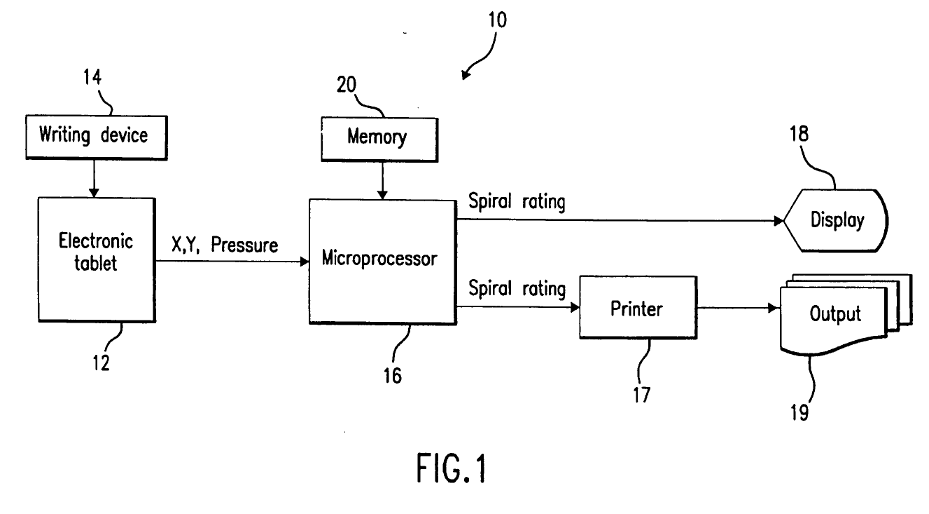

FIG. 1 shows a hardware block diagram of a system 10 for clinically assessing motor function in accordance with a preferred embodiment of the present invention. The system 10 includes an electronic digitizing tablet 12 having a writing device 14 for obtaining a geometric pattern handwritten by the subject and providing one or more signals representing the pattern, and a microprocessor 16 for processing the signals to derive one or more geometric indices representative of motor function and for computing from the indices, using an expert-generated “standard of reference,” a clinical rating score indicative of motor function.

The term “expert”, as used herein, refers to a person skilled in the assessment of motor function and/or in the diagnosis and assessment of one or more motor disorder. Non-limiting specific examples of suitable experts include physicians, preferably neurologists, and more preferably neurologists specialized in the field of motor disorders.

In a preferred embodiment of the present invention, the handwritten samples are freehand Archimedes spiral patterns, drawn on an electronic tablet 12, that are digitized and analyzed by the microprocessor 16 in accordance with a set of spiral indices shown to be indicative of motor function. Further as shown in FIG. 1, a display device 18 and/or printer 17 are provided for displaying and/or printing an output 19 of the clinical rating, geometric indices and other relevant information.

The system of FIG. 1 can be adapted, for example, for diagnosing and/or monitoring movement disorders such as Parkinson's disease, essential tremor and dystonia, for evaluating neurological development and handedness in children, and for rehabilitative purposes. The spiral analysis program is also capable of analyzing any motor disorder involving the upper limbs, e.g., hand, forearm, arm, shoulder. The system can also be adapted for handwriting identification and psychiatric evaluation purposes. With proper use of controls and normative data, spiral analysis should be of use in any condition from tremors to developmental abnormalities.

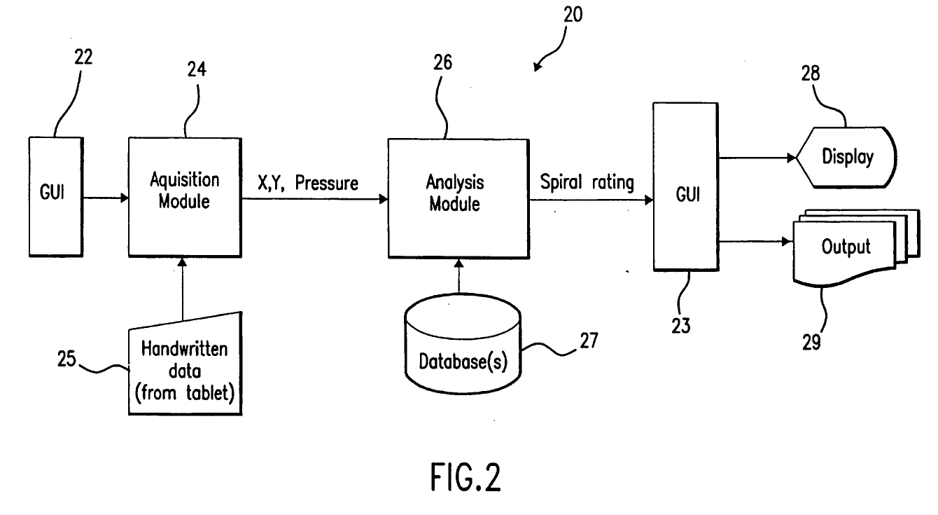

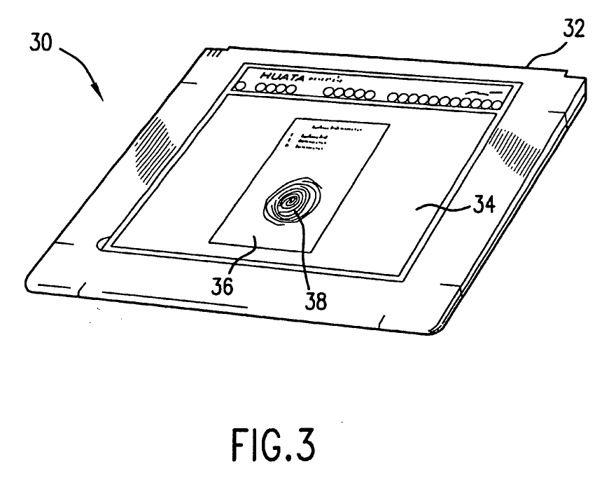

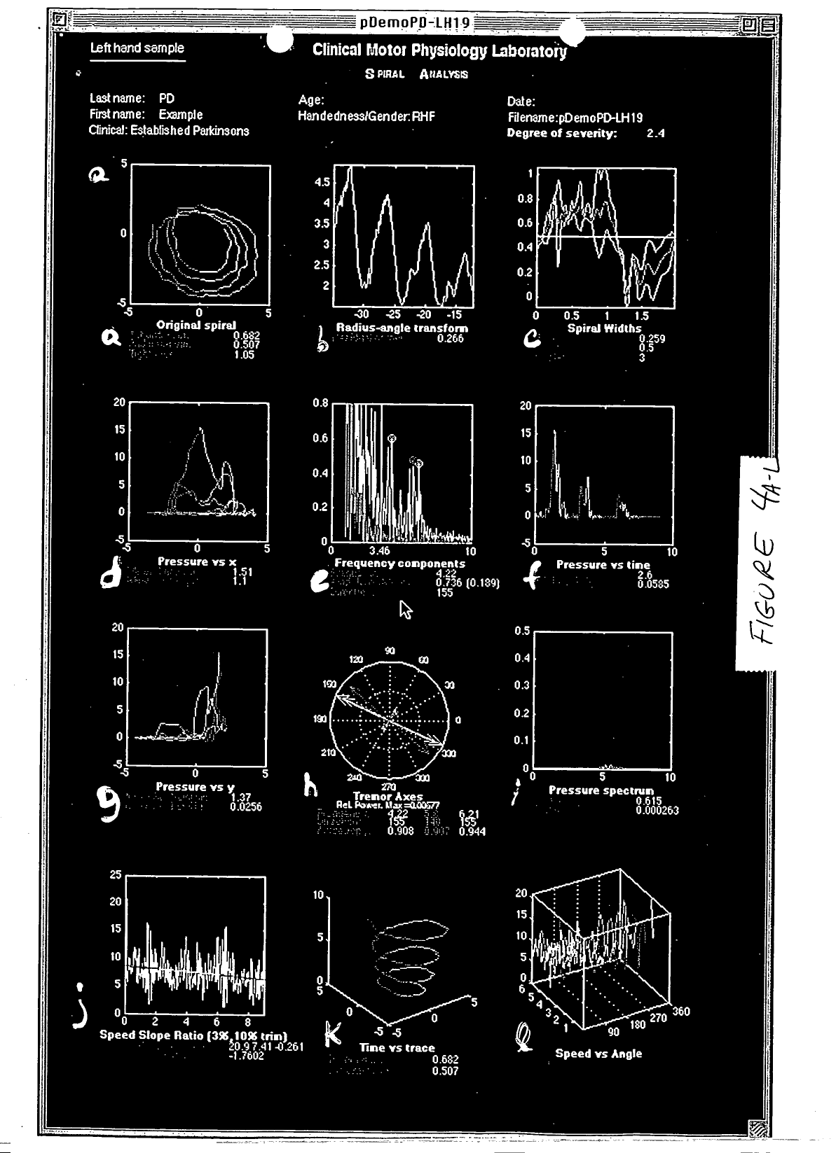

FIG. 2 shows a software block diagram corresponding to the system of FIG. 1. In a preferred embodiment of the present invention, the software 20 includes: an input graphical user interface (GUI) 22, an acquisition module 24, an analysis module 26, an analysis database 27 and an output GUI 23 which can deliver the results of analysis via a display device 28 and/or printer output 29. The acquisition module 24, via the GUI 22, instructs the user to provide any user-related information including user-defined parameters for generating a digitized geometric pattern. Handwritten “manual” data 25 is provided by the patient as instructed by the acquisition module. The X-position, Y-position and pressure data is then forwarded to the analysis module 26.

Referring again to FIG. 1, the data output from the electronic tablet is provided to the microprocessor 16, which is preferably an Apple MacIntosh or IBM-compatible personal computer. The microprocessor 16 is coupled to computer memory 20, which contains the analysis software module 26 shown in FIG. 2. The microprocessor 16 thus runs the analysis module 26, which in turn accesses an analysis database 27 (FIG. 2). The database 27 is used for storing and retrieving, for example, patient demographics and indices output. The analysis module 26 receives the X-position, Y-position and pressure data from the acquisition module 24 and computes a plurality of geometric indices used to assess the upper limb motor abilities of the patient. Although the analysis module 26 can be applied to analyze a variety of geometric patterns, the analysis module 26 of a preferred specific embodiment of the invention includes an algorithm that analyzes hand-drawn (Archimedean) spirals.

Additional information relating to the general practice of the invention, and the obtention of an expert-generated standard of reference, may be found in U.S. Pat. No. 6,454,706.

Formulae used to derive the indices of the invention are set forth below. Of note, the formula for calculating the “degree of severity” referred to herein is essentially the same as the “clinical rating score” referred to in U.S. Pat. No. 6,454,706. The score obtained as degree of severity correlates with the United Parkinson's Disease Rating Scale, established to rate the degree of severity of Parkinson's Disease and related disorders (see below). In the following equations, x and y are the Cartesian coordinates, α is a constant parameter, θ is an angle parameter, Θ is the total angular change, r=√(x²+y²), where γ=αθ translates the spiral into a linear relation; R is the total radius and K is the total number of points starting from 0.

1) DOS (Degree of Severity)
DOS=0.461486*Z₂−0.233112*Z₂²+0.253867*Z₂*dz2−0.072585*dz₂²−0.000970190*zd₂²+0.054407*zd+1.366767

Z₂—first order smoothness

dz₂—second order smoothness

zd—second order zero-crossing rate

2) Original Spiral (see FIG. 4a)

First Order Smoothness

$${z}_{2} = \frac{1}{Θ} \sum {( \frac{Δ r}{Δ θ} )}^{2} \vert Δ θ \vert$$

$${z}_{2} = ln {z}_{2}$$

Second Order Smoothness

$$d {z}_{2} = \frac{1}{Θ} \sum {( \frac{\frac{Δ r}{Δ θ}}{Δ θ} - d {\overline{r}}_{θ} )}^{2} \vert Δ θ \vert$$

$$d {z}_{2} = ln d {z}_{2}$$

$$Where d {\overline{r}}_{θ} is the average slope of \frac{Δ r}{Δ θ} \sim θ$$

First Order Zero Crossing Rate

$$\frac{1}{2 ( J - 1 )} {\sum }_{j = 1}^{J - 1} [ sign \{ {( \frac{Δ r}{Δθ} )}_{j + 1} - {( \frac{Δ r}{Δθ} )}_{{\overline{r}}_{θ}} \} ] - [ sign \{ {( \frac{Δ r}{Δθ} )}_{j} - {( \frac{Δ r}{Δθ} )}_{{\overline{r}}_{θ}} \} ] {Æ}_{} * 100 \%$$

$$where$$

$$J = Total number of data points in the series of acquired data \\ j = A specific data point in the series ( i . e . data point \#5 ) \\ {\sum }_{j = 1}^{J - 1} = Sum from first data point acquired ( j = 1 ) to the 2 nd to last \\ data point ( J - 1 ) . \\ {\overline{r}}_{θ} = The rms value of \frac{Δ r}{Δθ}$$

Note the “sign” function, which is essential to this calculation.
The output of the sign function has three values: 1, 0, or −1, depending on whether the value “x” (comprised of the terms

$${( \frac{Δ r}{Δθ} )}_{j + 1 \_or \_j} - {( \frac{Δ r}{Δθ} )}_{{\overline{r}}_{θ}}$$

in the equation) is greater than, equal to, or less than zero as follows:
sign(x)=1, if x>0
sign(x)=0, if x=0
sign(x)=−1, if x<0
   With the sign function:
If the change rate at a given point (either “j” or “j+1”) is greater than the average change rate, then sign {x>0} yields 1.
Conversely, if the change rate at that point is less than the average change rate, then sign {x<0}yields −1.
Second Order Zero Crossing Rate

$$\frac{1}{2 ( J - 1 )} {\sum }_{j = 1}^{J - 1} [ sign \{ ( \frac{\frac{Δ r}{Δθ}}{Δθ} ) \vert ( j + 1 ) - ( d {\overline{r}}_{θ} ) \} ] - [ sign \{ ( \frac{\frac{Δ r}{Δθ}}{Δθ} ) \vert ( j ) - ( d {\overline{r}}_{θ} ) \} ] {Æ}_{} * 100 \%$$

$$TIGHTNESS$$

$$Tightness value = \frac{{Θ}_{{actual}_{spiral}} / {R}_{{actual}_{spiral}}}{10 π / 10} \\ ts = ( \frac{θ}{R} - 10 π ) / 2 π$$

3) Radium-Angle Transform (see FIG. 4b)

Residual of theta-r

cti=(polyval*(ct,t(k);)−c(k))²

cti=sqrt(cti/d−2))

where r=sqrt(x²+y²)
c=atan2(y,x)
ct=polyfit*(t,c,n)
cti=sqrt(cti/d-2))
*MatLab function
   4) Spiral Width (see FIG. 4c)

Spiral width is defined as the distance between two neighboring points where the spiral curve intersects with a radial straight line. For a spiral of N cycles, there are N−1 measurements of spiral widths for one direction (angle). Since a spiral is represented by a set of discrete points, the intersecting points are computed using interpolation as follows:

Separate radius and angle data arrays into subarrays corresponding to each cycle
r₁(k)={r(k)|k=[kₛ, k₁]}, q₁(k)={q(k)|k=[kₛ, k₁]},
r₂(k)={r(k)|k=(k₁, k₂]}, q₂(k)={q(k)|k=[k₁, k₂]},
r₃(k)={r(k)|k=(k₂, kₑ]}, q₃(k)={q(k)|k=(k₂, kₑ]},
Where
0=<q₁(k)<2pi (1^{st} cycle)
2pi=<q₂(k)<4pi (2^{nd} cycle)
4pi=<q₃(k)<6pi (3^{rd} cycle)

Compute the points on the 2nd, 3rd, 4th, . . . cycles, which correspond to the angles given by q₁(k). MATLAB function to compute linear interpolation is used to calculate the radius of those points.
r′₂=interp1(r₂,q₂,q₁);
r′₃=interp1(r₃,q₃,q₁);
r′₄=interp1(r₄,q₄,q₁);

Compute the spiral widths between cycles
s₁=r′₂−r′₂
s₂=r′₃−r′₂
s₃=r′₄−r′₃

Statistics such as average, standard deviation, minimum, maximum, etc. can be computed from sᵢ, i=1, 2, 3, . . .

NOTE: Since the first cycle is used to determine the directions to measure the widths, it is necessary that spiral data is trimmed so that the first cycle is legitimate spiral.

See “slopeWidthsPlot.m” for actual computation.

5) Pressure vs. x (See FIG. 4d)

Determine the pressure data in Right and left side of spiral (see AnalySpiral.m)

$$Mean R hemi pr = \frac{{\sum }_{n = 1 : k} Rpr}{K + 1}$$

$$Mean R hemi pr = \frac{{\sum }_{n = 1 : k} Lpr}{K + 1}$$

Where Rpr pressure data in R hemi spiral
Lpr pressure data in L hemi spiral
   6) Frequency Components (See FIG. 4e)

Frequency of tremor
fx=fft(x1Taper2, m*4);

see “AnalySpiral.m”

Amplitude of tremor

Amp.Max (mean) Tremor amplitude is defined as the movement of the pen in the maximum tremor axis. The differences between neighboring extreme values in the tremor axis are averaged to determine the tremor amplitude as follows:

Rotate the spiral data (x,y) so that the tremor axis lies on the X axis so that x coordinate represent the movement in the tremor axis.
(x′,y′)->(x,y)

Determine the extermums eₓ(k) and compute the difference.
aₓ(k)=eₓ(k+1)−eₓ(k)

Remove the amplitudes above the cutoff to exclude the amplitude of the spiral itself.
a(k)=aₓ(k) where aₓ(k)<cutoff

Compute the minimum, maximum, and mean values of a(k)

See “compAmp.m”

Direction of tremor

d: angle (degree)

The angle, which shows the maximum power of tremor frequency

See “traxis.m”

7) Pressure vs. Time (See FIG. 4f)

Residue of pr-t (Pti)
pti=(polyval(pt,tt(k))−pp(k))ˆ2
pti=sqrt(pti/(d−2));

See “AnalySpiral.m”

8) Pressure vs. y (see FIG. 4g)
Mean R/L hemi pr=Mean R hemi pr/Mean L hemi pr
Mean R−L hemi pr=Mean R hemi pr−Mean L hemi pr

See “FIG. 2-1”

9) Tremor Axes (see FIG. 4h)

Rel. Power. Max (maxpower density of frequency f) and direction z(d)

P(f,d): (DFT{z(d)})²/N

z(d)=x cos(d pi/180)−y sin(d pi/180): x coordinates after rotated by d

N: length of z(d)

Direction
Pol=[P(f,dₘₐₓ(f))−P(f,dₘᵢₙ(f))]/[P(f,dₘₐₓ(f))+P(f,dₘᵢₙ(f))]

dₘₐₓ(f): direction where P(f,d) is maximum

dₘᵢₙ(f): direction where P(f,d) is minimum

10) Pressure Spectrum (see FIG. 4i)

Frequency
fp=fft(p1 Taper2,m*4)

Power
pfp=fp.*conj(fp)/(dˆ2)

See “AnalySpiral.m”

11) Speed Slope Ratio (See FIG. 4j)

Compute the moving speed of the pen by
v(t)=sqrt((dx/dt)²+(dx/dt)²)

Trim the beginning and the ending part from v(t)
vₜ(t)={v(t)|t=[tₛ,tₑ]}

Fit a second order curve to vₜ(t) in at least-squares sense

(MATLAB polyfit function is used)
vₜt˜=at²+bt+c=f(t)

Divide it into 3 segments,
f₁(t)={f(t)|t=[tₛ,t₁]}
f₂(t)={f(t)|t=[t₁,t₂]}
f₃(t)={f(t)|t=[t₂,tₑ]}
and compute fit line each segment in least-squares sense
f₁(t)˜=a₁t+b₁
f₂(t)˜=a₂t+b₂
f₃(t)˜=a₃t+b₃
slope1=a₁
slope2=a₂
slope3=a₃

Compute the ratio dividing the third slope by the abs of the first slope.
ratio=slope3/abs(slope1)

See “QPSpiAnaProind1csdMod.m” (at line 455%—option 2)

12) Time vs. Trace (See FIG. 4k)

13) Speed vs. Angle (See FIG. 41)

Program code indices (also see “AnalyAllData”)

index (n,k)

n: Trial Number

k: Index

| Listing of Indices | Description |
|---|---|
| 1. DegreeOfSeverity | degree of severity |
| 2. z2 | 1st order smoothness |
| 3. dz2 | 2nd order smoothness |
| 4. tx | Tightness |
| 5. z | |
| 6. zd | |
| 7. cti | |
| 8. pti | |
| 9. spi | |
| 10. MeanRightHemiPressure | |
| 11. MeanLeftHemiPressure | |
| 12. MeanRightDLeftHemiPressure | |
| 13. MeanRightMLeftHemiPressure | |
| 14. fx1 | |
| 15. abs(mfx1) | |
| 16. fy1 | |
| 17. abs(mfy1) | |
| 18. maxf | |
| 19. PressurePower | |
| 20. SpeedMax | |
| 21. SpeedMean | |
| 22. SpeedSlope | |
| 23. f1 | |
| 24. abs(mf1) | |
| 25. fc1 | |
| 26. abs(mfc1) | |
| Frequency, Direction, and Polarity | |
| 27. traxis_fr(1) | Frequency (Hz) |
| 28. traxis_dmax(1)*180/pi | Direction in degree |
| 29. traxis_pol(1) | Polarity |
| 30. traxis_fr(2) | |
| 31. traxis_dmax(2)*180/pi | |
| 32. traxis_pol(2) | |
| 33. traxis_fr(3) | |
| 34. traxis_dmax(3)*180/pi | |
| 35. traxis_pol(3) | |
| Power density of the Frequency | |
| detected | |
| 36. traxis_extra.pmax(1) | |
| 37. traxis_extra.pmax(2) | |
| 38. traxis_extra.pmax(3) | |
| Speed Slope | |
| 39. slope1(1) | Slope of the first segment |
| 40. slope2(1) | Slope of the second segment |
| 41. slope3(1) | Slope of the third segment |
| 42. ratio | Ratio of the slopes |
| Tremor Amplitude | |
| 43. maxAmp | Maximum of the amplitude |
| 44. meanAmp; | Mean of the amplitude |
| 45. stdAmp; | STD of the amplitude |

A Spiral Analysis Program is included herein as part of Appendix B, provided as a diskette.

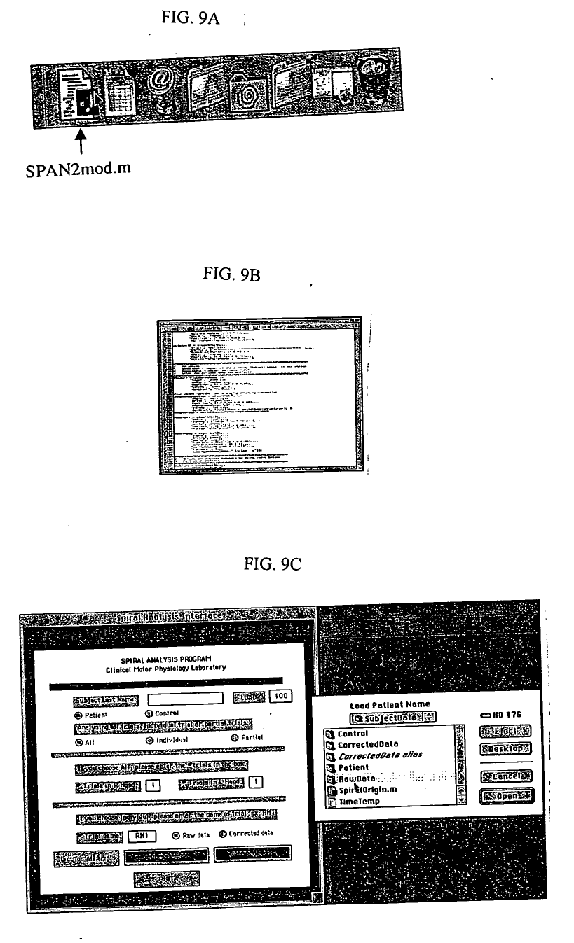

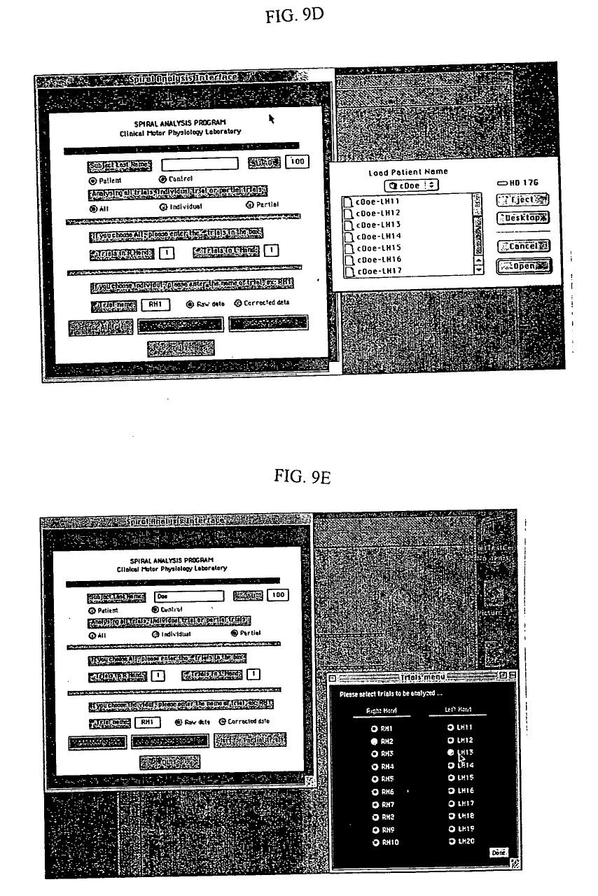

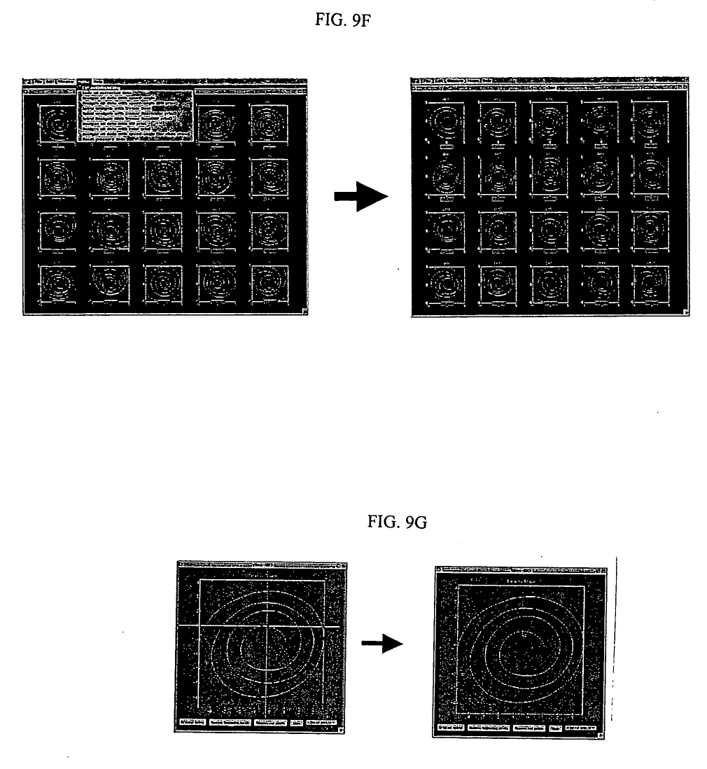

In a specific, non-limiting embodiment, the “Spiral Analysis” program may be opened by double clicking on a “SPAN2mod.m” shortcut in a menu (see FIG. 9A), which would open a window with programming code. To execute this code, Command+E may be hit simultaneously (see FIG. 9B). This may activate a “Spiral Analysis Interface.” Data from a subject of interest may then be loaded (see FIG. 9C). A subfolder containing “subject data” may contain further subfolders for “control”, “corrected data,” “raw data”, etc. Spirals may be analyzed individually or as a group, for example either a subset of, or all the spirals, collected during a drawing session (see FIG. 9D). In a specific non-limiting embodiment, spirals may be eliminated from a group analyzed by “deselecting” them (see FIG. 9E, where RH2 and LH13 are “unselected”). In another specific non-limiting embodiment (illustrated in FIG. 9F), the beginning and ending segments may be cut off of each spiral (excluding these data points from analysis) to make the spiral start and end on the same horizontal plane; the beginning and/or end segments may be cut further, for example (see FIG. 9G), using a system in which cross-hairs appear on the screen which allow a portion or portions of a spiral which are to be trimmed identified (the system may retain a copy of the original spiral). The data may then be analyzed.

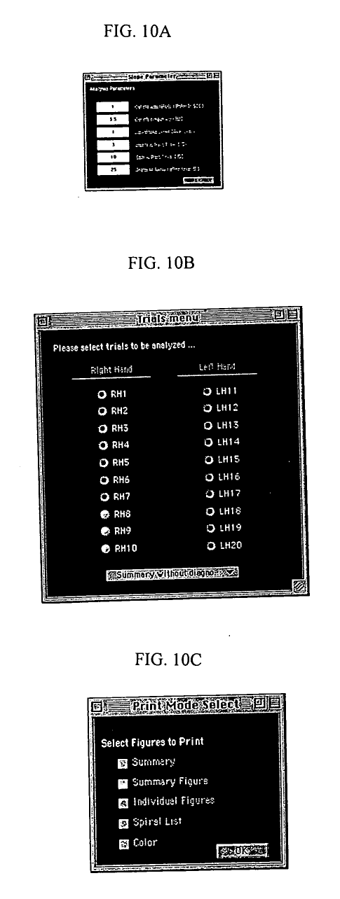

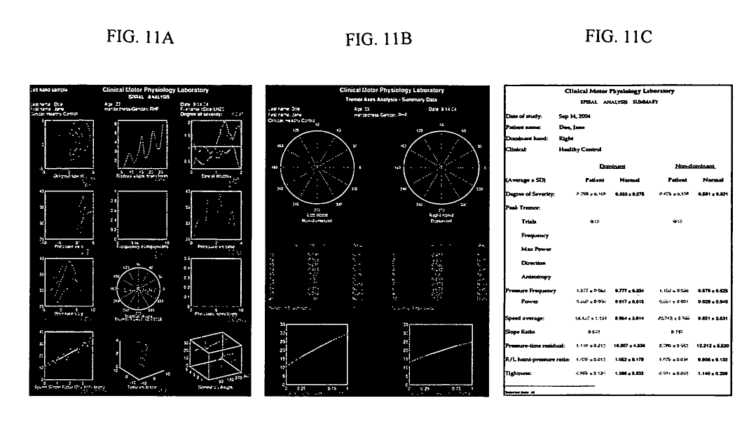

In a specific, non-limiting embodiment, as depicted in FIG. 10A-C, an analysis option may be selected depending on the desired printing of the data, for example, with selections “Menu ‡ Spiral Analysis with Tremor Axes (Color Print)”; “Menu ‡ Spiral Analysis with Tremor Axes (BW Print)” (which may be less easy to read); “Menu ‡ Spiral Analysis With Tremor Axes” which does not automatically print. After selecting one of these options, a pop-up box may appear with several parameters/values as shown in FIG. 10A, were “OK” may be clicked. As shown in FIG. 10B, after analysis of all 20 individual spirals is complete, a box may appear asking if the operator would like a Spiral Analysis Summary. If “Yes” is selected, a Trials Menu box may appear. Because it is generally desirable to obtain a summary sheet that reflects data from all the analyzed spirals, it may be desirable not to unselect any of the individual spirals. In this specific example, if one clicks on “Summary Without Diagnosis” until the text flashes, a Summary Sheet Window may appear. Again, in this non-limiting illustrative example (see FIG. 10C), the last window which may be encountered may offer another opportunity to print any combination of analysis output windows. If “Spiral Analysis with Tremor Axes” with “Color Print” or “BW Print” had been selected, printing may have already commenced. It may be desirable to click “OK” to print out “Summary” and “Spiral List” as well. FIG. 11A-C shows various options of printed output.

### Uses of the Invention

One object of the present invention is to provide a relatively inexpensive and non-invasive computerized system and method for clinically assessing motor function. Such a system and method can be adapted for analyzing movement disorders such as Parkinson's disease, essential tremor and dystonia, and for characterizing neurological development and handedness in children. It may be used to identify visual field defects or neglect, migraine, epilepsy, ischemic injury, psychiatric disorders (e.g., perseverative behavior), or drug toxicity (e.g. drug induced Parkinsonism). It may be used in the context of clinical practice of medicine, or in the context of research directed toward the motor systems and treatments for disorders thereof.

In particular embodiments, the present invention relates to a computerized system and method for clinically assessing motor function comprising correlating spiral indices, computed from digital information obtained from a spiral shape drawn by a subject to be evaluated, with a clinical rating score derived using a “standard of reference” generated by one or more clinical expert. By analogy to a biochemical assay, which measures the amount of reactant by comparison to a standard curve, the present invention provides a method and system by which a medical practitioner can evaluate the motor function of a subject by generating a digitized writing sample and computationally comparing geometric indices obtained therefrom with values associated with clinical ratings assigned by skilled neurologists. Interpretation is thereby rendered more objective and consistent. Furthermore, the test may be administered and interpreted by physicians who are not skilled or experienced in evaluating motor disorders, for example general practitioners or pediatricians who are not specialized in the practice of neurology. The present invention therefore provides a means for evaluating persons early in the course of disease, and for screening patients for motor dysfunction or, in the case of children, disorders of motor development.

In the research context, an index or indices or depiction thereof obtained using the present invention may be used in evaluations of the effects of pharmacologic or surgical intervention on motor function, and as such may be used in conjunction with either an expert generated standard or, alternatively, using internal comparison(s). This may be done in a clinical practice or research context.

As one non-limiting specific example, the present invention provides for indices and depictions thereof that relate to frequency components of tremor and tremor axes. Comparison of such indices and depictions with expert-generated standards of reference may be used to distinguish tremor resulting from Parkinsonism or from familial essential tremor. Alternatively, an index, indices and/or depictions thereof may be obtained for a particular subject, and then the subject may be reevaluated after the passage of time and/or the administration of an agent, and the later index/indices may be compared with the earlier to evaluate disease progression or therapeutic benefit, respectively, without an expert-generated standard of reference involved.

Accordingly, in one set of embodiments, the present invention provides for a method for assessing motor function in a subject comprising:

(i) obtaining, from the subject, a handwritten spiral that is converted into representative digital information;

(ii) calculating, from the digital information obtained in step (i), at least one index selected from the group consisting of speed by angle over 360°, mean spacing between spiral loops, by angle over 360°, confidence intervals of spiral loop spacing, number of spiral loops, X-Y plane frequency spectrum, X-Y plane frequency power, X-Y tremor peak angular direction, X-Y tremor anisotropy, peak spiral speed, speed slope ratios between first and last segments, speed variability between first and last segments, and acceleration residuals between first and last segments; and

(iii) comparing the index or indices calculated by step (ii) with a control value for each index, where the control value may be a value obtained from the same subject at a different point in time, a value obtained from a subject having normal motor function, a value obtained from a subject having a motor disorder, or an expert-generated standard of reference. The difference between the indices calculated for the subject's spiral with control values provides an assessment of motor function. The aforelisted indices may be used in addition to a calculation of degree of severity, as set forth below and as described in U.S. Pat. No. 6,454,706 (where it is referred to as “clinical rating score,”) to assess motor function.

Alternatively, the present invention, in other embodiments, provides for a method for determining whether a test agent has an effect on motor function in a subject, comprising:

(i) administering the test agent to the subject;

(ii) obtaining, from the subject, a handwritten spiral that is converted into representative digital information;

(iii) calculating, from the digital information obtained in step (i), at least one index selected from the group consisting of speed by angle over 360°, mean spacing between spiral loops, by angle over 360°, confidence intervals of spiral loop spacing, number of spiral loops, X-Y plane frequency spectrum, X-Y plane frequency power, X-Y tremor peak angular direction, X-Y tremor anisotropy, peak spiral speed, speed slope ratios between first and last segments, speed variability between first and last segments, and acceleration residuals between first and last segments; and

(iv) comparing the index or indices calculated by step (ii) with a control value for each index, where the control value may be a value obtained from the subject in the absence of administration of test agent, a value obtained from a subject having normal motor function, a value obtained from a subject having a motor disorder, or an expert generated standard of reference. A difference between the subject's spiral indices after administration of the test agent and control values indicates that the test agent modulates motor function.

The foregoing indices may further be combined, for the assessment, with one or more of the following indices (disclosed in U.S. Pat. No. 6,454,706): first order smoothness, second order smoothness, tightness of loops, first order “zero” crossing rate, second order “zero” crossing rate, residue of radius-angle regression (second order polynomial, least square), residue of time-pressure regression (second order polynomial, least square), residue of speed-time regression, X-axis frequency (dominant), dominant x-axis frequency power, Y-axis frequency (dominant), dominant y-axis frequency power, angular velocity frequency, dominant angular speed frequency power, X-Y combined speed frequency power (dominant), dominant X-Y combined speed frequency power, or residue of angular velocity-time regression, or with a calculation of degree of severity.

The present invention further provides for a method for assessing motor function in a subject comprising obtaining indices as set forth above and then graphically presenting the data as one or more graphs selected from the group consisting of spiral widths, frequency components, tremor axes, speed/slope ratio and speed versus angle (see FIG. 4).

### Example: Sample Evaluation

A depiction of data obtained from a subject is set forth in FIG. 4.

### Unified Parkinson's Disease Rating Scale

(from http://www.parkinson.org/site/pp.asp?c=9dJFJLPwB&b=123510) The UPDRS is a rating tool to follow the longitudinal course of Parkinson's Disease. It is made up of the 1) Mentation, Behavior, and Mood, 2) ADL and 3) Motor sections. These are evaluated by interview. Some sections require multiple grades assigned to each extremity. A total of 199 points are possible. 199 represents the worst (total) disability), 0—no disability.

I. Mentation, Behavior, Mood

Intellectual Impairment

0—none

1—mild (consistent forgetfulness with partial recollection of events with no other difficulties)

2—moderate memory loss with disorientation and moderate difficulty handling complex problems

3—severe memory loss with disorientation to time and often place, severe impairment with problems

4—severe memory loss with orientation only to person, unable to make judgments or solve problems

Thought Disorder

0—none

1—vivid dreaming

2—“benign” hallucination with insight retained

3—occasional to frequent hallucination or delusions without insight, could interfere with daily activities

4—persistent hallucination, delusions, or florid psychosis.

Depression

0—not present

1—periods of sadness or guilt greater than normal, never sustained for more than a few days or a week

2—sustained depression for >1 week

3—vegetative symptoms (insomnia, anorexia, abulia, weight loss)

4—vegetative symptoms with suicidality

Motivation/Initiative

0—normal

1—less of assertive, more passive

2—loss of initiative or disinterest in elective activities

3—loss of initiative or disinterest in day to say (routine) activities

4—withdrawn, complete loss of motivation

II. Activities of Daily Living

Speech

0—normal

1—mildly affected, no difficulty being understood

2—moderately affected, may be asked to repeat

3—severely affected, frequently asked to repeat

4—unintelligible most of time

Salivation

0—normal

1—slight but noticeable increase, may have nighttime drooling

2—moderately excessive saliva, hay minimal drooling

3—marked drooling

Swallowing

0—normal

1—rare choking

2—occasional choking

3—requires soft food

4—requires NG tube or G-tube

Handwriting

0—normal

1—slightly small or slow

2—all words small but legible

3—severely affected, not all words legible

4—majority illegible

Cutting Food/Handing Utensils

0—normal

1—somewhat slow and clumsy but no help needed

2—can cut most foods, some help needed

3—food must be cut, but can feed self

4—needs to be fed

Dressing

0—normal

1—somewhat slow, no help needed

2—occasional help with buttons or arms in sleeves

3—considerable help required but can do something alone

4—helpless

Hygiene

0—normal

1—somewhat slow but no help needed

2—needs help with shower or bath or very slow in hygienic care

3—requires assistance for washing, brushing teeth, going to bathroom

4—helpless

Turning in Bed/Adjusting Bed Clothes

0—normal

1—somewhat slow no help needed

2—can turn alone or adjust sheets but with great difficulty

3—san initiate but not turn or adjust alone

4—helpless

Falling-Unrelated to Freezing

0—none

1—rare falls

2—occasional, less than one per day

3—average of once per day

4—>1 per day

Freezing When Walking

0—normal

1—rare, may have start hesitation

2—occasional falls from freezing

3—frequent freezing, occasional falls

4—frequent falls from freezing

Walking

0—normal

1—mild difficulty, day drag legs or decrease arm swing

2—moderate difficultly requires no assist

3—severe disturbance requires assistance

4—cannot walk at all even with assist

Tremor

0—absent

1—slight and infrequent, not bothersome to patient

2—moderate, bothersome to patient

3—severe, interfere with many activities

4—marked, interferes with many activities

Sensory Complaints Related to Parkinsonism

0—none

1—occasionally has numbness, tingling, and mild aching

2—frequent, but not distressing

3—frequent painful sensation

4—excruciating pain

III. Motor Exam

Speech

0—normal

1—slight loss of expression, diction, volume

2—monotone, slurred but understandable, mod. impaired

3—marked impairment, difficult to understand

4—unintelligible

Facial Expression

0—Normal

1—slight hypomymia, could be poker face

2—slight but definite abnormal diminution in expression

3—mod. hypomimia, lips parted some of time

4—masked or fixed face, lips parted ¼ of inch or more with complete loss of expression

*Tremor at Rest

Face

0—absent

1—slight and infrequent

2—mild and present most of time

3—moderate and present most of time

4—marked and present most of time

Right Upper Extremity (RUE)

0—absent

1—slight and infrequent

2—mild and present most of time

3—moderate and present most of time

4—marked and present most of time

LUE

0—absent

1—slight and infrequent

2—mild and present most of time

3—moderate and present most of time

4—marked and present most of time

RLE

0—absent

1—slight and infrequent

2—mild and present most of time

3—moderate and present most of time

4—marked and present most of time

LLE

0—absent

1—slight and infrequent

2—mild and present most of time

3—moderate and present most of time

4—marked and present most of time

*Action or Postural Tremor

RUE

0—absent

1—slight, present with action

2—moderate, present with action

3—moderate present with action and posture holding

4—marked, interferes with feeding

LUE

0—absent

1—slight, present with action

2—moderate, present with action

3—moderate present with action and posture holding

4—marked, interferes with feeding

*Rigidity

Neck

0—absent

1—slight or only with activation

2—mild/moderate

3—marked, full range of motion

4—severe

RUE

0—absent

1—slight or only with activation

2—mild/moderate

3—marked, full range of motion

4—severe

LUE

0—absent

1—slight or only with activation

2—mild/moderate

3—marked, full range of motion

4—severe

RLE

0—absent

1—slight or only with activation

2—mild/moderate

3—marked, full range of motion

4—severe

LLE

0—absent

1—slight or only with activation

2—mild/moderate

3—marked, full range of motion

4—severe

*Finger taps

Right

0—normal

1—mild slowing, and/or reduction in amp.

2—moderate impaired. Definite and early fatiguing, may have occasional arrests

3—severely impaired. Frequent hesitations and arrests.

4—can barely perform

Left

0—normal

1—mild slowing, and/or reduction in amp.

2—moderate impaired. Definite and early fatiguing, may have occasional arrests

3—severely impaired. Frequent hesitations and arrests.

4—can barely perform

*Hand Movements (open and close hands in rapid succession)

Right

0—normal

1—mild slowing, and/or reduction in amp.

2—moderate impaired. Definite and early fatiguing, may have occasional arrests

3—severely impaired. Frequent hesitations and arrests.

4—can barely perform

Left

0—normal

1—mild slowing, and/or reduction in amp.

2—moderate impaired. Definite and early fatiguing, may have occasional arrests

3—severely impaired. Frequent hesitations and arrests.

4—can barely perform

*Rapid Alternating Movements (pronate and supinate hands)

Right

0—normal

1—mild slowing, and/or reduction in amp.

2—moderate impaired. Definite and early fatiguing, may have occasional arrests

3—severely impaired. Frequent hesitations and arrests.

4—can barely perform

Left

0—normal

1—mild slowing, and/or reduction in amp.

2—moderate impaired. Definite and early fatiguing, may have occasional arrests

3—severely impaired. Frequent hesitations and arrests.

4—can barely perform

*Leg Agility (tap heel on ground, amp should be 3 inches)

Right

0—normal

1—mild slowing, and/or reduction in amp.

2—moderate impaired. Definite and early fatiguing, may have occasional arrests

3—severely impaired. Frequent hesitations and arrests.

4—can barely perform

Left

0—normal

1—mild slowing, and/or reduction in amp.

2—moderate impaired. Definite and early fatiguing, may have occasional arrests

3—severely impaired. Frequent hesitations and arrests.

4—can barely perform

*Arising From Chair (pt. arises with arms folded across chest)

0—normal

1—slow, may need more than one attempt

2—pushes self up from arms or seat

3—tends to fall back, may need multiple tries but can arise without assistance

4—unable to arise without help

*Posture

0—normal erect

1—slightly stooped, could be normal for older person

2—definitely abnormal, mod. stooped, may lean to one side

3—severely stooped with kyphosis

4—marked flexion with extreme abnormality of posture

*Gait

0—normal

1—walks slowly, may shuffle with short steps, no Destination or propulsion

2—walks with difficulty, little or no assistance, some Destination, short steps or propulsion

3—severe disturbance, frequent assistance

4—cannot walk

*Postural Stability (retropulsion test)

0—normal

1—recovers unaided

2—would fall if not caught

3—falls spontaneously

4—unable to stand

*Body Bradykinesia/Hypokinesia

0—none

1—minimal slowness, could be normal, deliberate character

2—mild slowness and poverty of movement, definitely abnormal, or dec. amp. of movement

3—moderate slowness, poverty, or small amplitude

4—marked slowness, poverty, or amplitude.

Various publications are cited herein, the contents of which are hereby incorporated by reference in their entireties.

What is claimed is:

1. A method for assessing motor function in a subject comprising:
(i) obtaining, from the subject, a handwritten spiral that is converted into representative digital information;
(ii) calculating, from the digital information obtained in step (i), at least one index selected from the group consisting of speed by angle over 360°, mean spacing between spiral loops, by angle over 360°, confidence intervals of spiral loop spacing, number of spiral loops, X-Y plane frequency spectrum, X-Y plane frequency power, X-Y tremor peak angular direction, X-Y tremor anisotropy, peak spiral speed, speed slope ratios between first and last segments, speed variability between first and last segments, and acceleration residuals between first and last segments; and
(iii) comparing the index or indices calculated by step (ii) with at least one control value,
wherein the relative value of the index or indices with the control value or values provides an assessment of the motor function of the subject.

2. The method of claim 1, wherein a control value is an expert generated standard of reference.

3. The method of claim 1, wherein at least one control value is a value obtained from the same subject at a different point in time.

4. The method of claim 1, wherein at least one control value is a value obtained from a subject having normal motor function.

5. The method of claim 1, wherein at least one control value is a value obtained from a subject having a motor disorder.

6. A method for determining whether a test agent has an effect on motor function in a subject, comprising:
(i) administering the test agent to the subject;
(ii) obtaining, from the subject, a handwritten spiral that is converted into representative digital information;
(iii) calculating, from the digital information obtained in step (i), at least one index selected from the group consisting of speed by angle over 360°, mean spacing between spiral loops, by angle over 360°, confidence intervals of spiral loop spacing, number of spiral loops, X-Y plane frequency spectrum, X-Y plane frequency power, X-Y tremor peak angular direction, X-Y tremor anisotropy, peak spiral speed, speed slope ratios between first and last segments, speed variability between first and last segments, and acceleration residuals between first and last segments; and
(iv) comparing the index or indices calculated by step (ii) with at least one control value;
wherein a difference between the index or indices calculated in step (ii) and the control value indicates that the test agent modulates motor function.

7. The method of claim 6, wherein at least one control value is a value obtained from the subject in the absence of administration of test agent.

8. The method of claim 6, wherein at least one control value is a value obtained from a subject having normal motor function.

9. The method of claim 6, wherein at least one control value is a value obtained from a subject having a motor disorder.

10. The method of claim 6, wherein at least one control value is an expert generated standard of reference.

11. A method for assessing motor function in a subject comprising:
(i) obtaining, from the subject, a handwritten spiral that is converted into representative digital information;
(ii) calculating, from the digital information obtained in step (i), at least one index selected from the group consisting of speed by angle over 360°, mean spacing between spiral loops, by angle over 360°, confidence intervals of spiral loop spacing, number of spiral loops, X-Y plane frequency spectrum, X-Y plane frequency power, X-Y tremor peak angular direction, X-Y tremor anisotropy, peak spiral speed, speed slope ratios between first and last segments, speed variability between first and last segments, and acceleration residuals between first and last segments and at least one index selected from the group consisting of first order smoothness, second order smoothness, tightness of loops, first order “zero” crossing rate, second order “zero” crossing rate, residue of radius-angle regression (second order polynomial, least square), residue of time-pressure regression (second order polynomial, least square), residue of speed-time regression, X-axis frequency (dominant), dominant x-axis frequency power, Y-axis frequency (dominant), dominant y-axis frequency power, angular velocity frequency, dominant angular speed frequency power, X-Y combined speed frequency power (dominant), dominant X-Y combined speed frequency power, or residue of angular velocity-time regression.; and
(iii) comparing the indices calculated by step (ii) with control values,
wherein the relative value of the indices with the control values provides an assessment of the motor function of the subject.

12. The method of claim 11, wherein at least one control value is an expert generated standard of reference.

13. The method of claim 11, wherein at least one control value is a value obtained from the same subject at a different point in time.

14. The method of claim 11, wherein at least one control value is a value obtained from a subject having normal motor function.

15. The method of claim 11, wherein at least one control value is a value obtained from a subject having a motor disorder.

16. A method for determining whether a test agent has an effect on motor function in a subject, comprising:
(i) administering the test agent to the subject;
(ii) obtaining, from the subject, a handwritten spiral that is converted into representative digital information;
(iii) calculating, from the digital information obtained in step (i), at least one index selected from the group consisting of speed by angle over 360°, mean spacing between spiral loops, by angle over 360°, confidence intervals of spiral loop spacing, number of spiral loops, X-Y plane frequency spectrum, X-Y plane frequency power, X-Y tremor peak angular direction, X-Y tremor anisotropy, peak spiral speed, speed slope ratios between first and last segments, speed variability between first and last segments, and acceleration residuals between first and last segments and at least one index selected from the group consisting of first order smoothness, second order smoothness, tightness of loops, first order “zero” crossing rate, second order “zero” crossing rate, residue of radius-angle regression (second order polynomial, least square), residue of time-pressure regression (second order polynomial, least square), residue of speed-time regression, X-axis frequency (dominant), dominant x-axis frequency power, Y-axis frequency (dominant), dominant y-axis frequency power, angular velocity frequency, dominant angular speed frequency power, X-Y combined speed frequency power (dominant), dominant X-Y combined speed frequency power, or residue of angular velocity-time regression; and
(iv) comparing the indices calculated by step (iii) with control indices;
wherein a difference between the indices calculated by step (iii) and control indices indicates that the test agent modulates motor function.

17. The method of claim 16, wherein at least one control value is a value obtained from the subject in the absence of administration of test agent.

18. The method of claim 16, wherein at least one control value is a value obtained from a subject having normal motor function.

19. The method of claim 16, wherein at least one control value is a value obtained from a subject having a motor disorder.

20. The method of claim 16, wherein at least one control value is an expert generated standard of reference.

21. A method for assessing tremor in a subject comprising:
(i) obtaining, from the subject, a handwritten spiral that is converted into representative digital information;
(ii) calculating, from the digital information obtained in step (i), at least one index selected from the group consisting of X-Y tremor peak angular direction and X-Y tremor anisotropy, and
(iii) comparing the indices calculated by step (ii) with control values,
wherein the relative value of the indices with the control values provides an assessment of the motor function of the subject.

22. A method for assessing motor function in a subject comprising obtaining indices as set forth in claim 1, 6, 11, 16 or 21, and then graphically presenting the data as one or more graphs selected from the group consisting of spiral widths, frequency components, tremor axes, speed/slope ratio and speed versus angle.

23. A method for assessing tremor in a subject comprising obtaining indices as set forth in claim 1, 6, 11, 16 or 21, and then graphically presenting the data as one or more graphs selected from the group consisting of frequency components and tremor axes.

24. A digitized tablet programmed to perform the method of claim 1.
